(19) United States
(12) Patent Application Publication
(10) Pub. No.: US 2009/0297540 A1
(43) Pub. Date: Dec. 3, 2009

(54) Induction of Indoleamine 2,3-Dioxygenase in Dendritic Cells by TLR Ligands and Uses thereof

(76) Inventors: Andrew Mellor, Augusta, GA (US); David Munn, Augusta, GA (US)

Correspondence Address: MUETING, RAASCH & GEBHARDT, P.A., P.O. BOX 581336, MINNEAPOLIS, MN, 55458-1336, US

(21) Appl. No.: 12/083,855

(22) PCT Filed: Oct. 20, 2006
(86) PCT No.: PCT/US06/40796

§ 371 Date: Jul. 20, 2009

Related U.S. Application Data
(60) Provisional application No. 60/729,041, filed on Oct. 21, 2005.

(51) Int. Cl. A61K 39/00; A61K 45/00; C12N 5/06; A61K 31/7088; A61P 37/04

(52) U.S. Cl. 424/184.1; 424/93.7; 435/325; 514/44 R

(57) ABSTRACT

The induction of indoleamine 2,3-dioxygenase (IDO) in an IDO-competent subset of dendritic cells by TLR ligands, including TLR9 ligands, and various uses thereof are presented.

### GOVERNMENT FUNDING

The present invention was made with government support under Grant Nos. HD41187, AI063402, CA103320, and CA096651 awarded by the National Institutes of Health. The Government may have certain rights in this invention.

### BACKGROUND

A newly recognized molecular mechanism contributing to peripheral immune tolerance is the immunoregulatory enzyme indoleamine 2,3-dioxygenase (IDO). Cells expressing the tryptophan-catabolizing enzyme IDO are capable of inhibiting T cell proliferation in vitro and reducing T cell immune responses in vivo (U.S. Pat. Nos. 6,451,840 and 6,482,416; Munn et al., Science 1998; 281:1191; Munn et al., J. Exp. Med. 1999; 189:1363; Hwu et al., J. Immunol. 2000; 164:3596; Mellor et al., J. Immunol. 2002; 168:3771; Grohmann et al., J. Immunol. 2001; 167:708; Grohmann et al., J. Immunol. 2001; 166:277; and Alexander et al., Diabetes 2002; 51:356).

IDO degrades the essential amino acid tryptophan (for reviews see Taylor et al., FASEB Journal 1991; 5:2516-2522; Lee et al., Laboratory Investigation, 2003; 83:1457-1466; and Grohmann et al., Trends in Immunology 2003; 24:242-248). Expression of IDO by human monocyte-derived macrophages (Munn et al., J. Exp. Med. 1999; 189:1363-1372), human dendritic cells (Munn et al., Science 2002; 297:1867-1870 and Hwu et al., J. Immunol. 2000; 164:3596-3599), and mouse dendritic cells (Mellor et al., J. Immunol. 2003; 171:1652-1655) allows these different antigen-presenting cells (APCs) to inhibit T cell proliferation in vitro. In vivo, IDO participates in maintaining maternal tolerance toward the antigenically foreign fetus during pregnancy (Munn et al., Science 1998; 281:1191-1193).

IDO has also been implicated in maintaining tolerance to self antigens (Grohmann et al., J. Exp. Med. 2003; 198:153-160), in suppressing T cell responses to MHC-mismatched organ transplants (Miki et al., Transplantation Proceedings 2001; 33:129-130; Swanson, et al. Am J Respir Cell Mol Biol 2004; 30:311-8; Beutelspacher et al. Am J Transplant 2006; 6:1320-30) and in the tolerance-inducing activity of recombinant CTLA4-Ig (Grohmann et al. Nature Immunology 2002; 3:985-1109; Mellor et al. J. Immunol 2003; 171:1652-1655) and the T cell regulatory functions of interferons (Grohmann et al. J Immunol 2001; 167:708-14; and Baban et al. Int. Immunol 2005; 17:909-919). In these four systems, the immunosuppressive effect of IDO can be blocked by the in vivo administration of an IDO inhibitor, such as 1-methyl-tryptophan (also referred to herein as 1-MT or 1MT).

The transfection of IDO into mouse tumor cell lines confers the ability to suppress T cell responses both in vitro and in vivo (Mellor et al., J. Immunol. 2002; 168:3771-3776). In a Lewis Lung carcinoma (LLC) model, administration of 1-MT significantly delayed tumor outgrowth (Friberg et al., International Journal of Cancer 2002; 101:151-155). The mouse mastocytoma tumor cell line forms lethal tumors in naive hosts, but is normally rejected by pre-immunized hosts. However, transfection of P815 with IDO prevents its rejection by pre-immunized hosts (Uyttenhove et al., Nature Medicine 2003; 9:1269-1274). Inhibition of tumor growth was entirely dependent on the presence of an intact immune system and was substantially reversed, that is, tumor growth inhibited, by the concomitant administration of 1-MT.

The selective recruitment of IDO⁺ APCs in the tumor-draining (sentinel) lymph nodes of patients with melanoma (Munn et al., Science 2002; 297:1867-1870 and Lee et al., Laboratory Investigation 2003; 83:1457-1466) indicates that tumors take advantage of the immunosuppressive effect of IDO by recruiting a population of IDO-expressing host APCs to present tumor antigens. Similar changes have been seen in breast carcinoma and other tumor-associated lymph nodes. In mouse tumor models the IDO-expressing APCs in tumor-draining lymph nodes are phenotypically similar to a subset of dendritic cells recently shown to mediate profound IDO-dependent immunosuppressive in vivo (Mellor et al., J. Immunol. 2003; 171:1652-1655; and Baban et al. Int. Immunol 2005; 17:909-919). IDO-expressing APCs in tumor-draining lymph nodes thus constitute a potent tolerogenic mechanism.

### SUMMARY OF THE INVENTION

The present invention includes a method of enhancing an immune response in a subject, the method including administering to the subject a TLR9 agonist and an inhibitor of indoleamine 2,3-dioxygenase (IDO).

The present invention also includes a method of enhancing an immune response to an antigen in a subject, the method including administering to the subject a TLR9 agonist, the antigen and an inhibitor of IDO.

The present invention also includes a method of stimulating CD8 T cell activation in a subject, the method including administering to the subject a TLR9 agonist and an inhibitor of IDO.

The present invention also includes a method of treating cancer in a subject, the method including administering to the subject a TLR9 agonist and inhibitor of IDO.

The present invention also includes a method of treating cancer in a subject, the method including administering to the subject a TLR9 agonist, an inhibitor of IDO, and an additional therapeutic agent.

Also included in the present invention are methods of suppressing a T cell mediated immune response in a subject, the method including administering to the subject a TLR9 agonist in an amount effective to induce indoleamine 2,3-dioxygenase (IDO) expression in an IDO-competent subset of dendritic cells.

Also included in the present invention is a method of inducing indoleamine 2,3-dioxygenase (IDO) expression in dendritic cells in a subject, the method including administering to the subject a TLR9 agonist in an amount effective to induce IDO expression in an IDO-competent subset of dendritic cells.

The present invention also includes a method of regulating a T cell mediated immune response in a subject, the method including administering to the subject a TLR9 agonist in an amount effective to induce IDO expression in an IDO-competent subset of dendritic cells.

The present invention also includes a method of suppressing T cell activation to an antigen in a subject, the method including administering to the subject a TLR9 agonist and the antigen, wherein the TLR9 agonist is administered in an amount effective to induce IDO expression in an IDO-competent subset of dendritic cells. In some embodiments of the method, the TLR9 agonist and the antigen are administered simultaneously. In some embodiments, the TLR9 agonist may be administered prior to the administration of the antigen. In some embodiments, the antigen may be an alloantigen.

The invention also includes a method of inducing tolerance to an antigen in a subject, the method including administering to the subject a TLR9 agonist and the antigen, wherein the TLR9 agonist is administered in an amount effective to induce IDO expression in an IDO-competent subset of dendritic cells.

The invention also includes a method of preventing allograft rejection in a subject, the method including administering to the subject a TLR9 agonist and one or more alloantigens present in the allograft.

The invention also includes a method of preventing allograft rejection in a recipient, the method including administering a TLR9 agonist to the recipient after the transplantation of the allograft into the recipient.

The invention also includes a method of preventing graft versus host disease in a recipient, the method including: administering to the donor a TLR9 agonist and one or more alloantigens present in the recipient, wherein the TLR9 agonist and the one or more alloantigens present in the recipient are administered to the donor prior to obtaining donor cells from the donor; obtaining donor cells from the donor; and administering the donor cells to the recipient.

The invention also includes a method of preconditioning a recipient of an allograft to suppress allograft rejection in the recipient, the method including: administering to the recipient a TLR9 agonist and one or more alloantigens present in the allograft, wherein the TLR9 agonist and the one or more alloantigens present in the allograft are administered to the recipient prior to allografting; and transplanting the allograft into the recipient.

With any of the methods of the present invention, the TLR9 agonist may be a CpG-oligonucleotide.

With any of the methods of the present invention, the TLR9 agonist may be administered systemically.

Also included in the present invention are isolated cell populations preconditioned to minimize graft versus host disease when transplanted into a donor recipient, the cell population obtained by a method including: administering to the donor a TLR9 agonist and one or more alloantigens present in the recipient, wherein the TLR9 agonist and the one or more alloantigens present in the recipient are administered to the donor prior to obtaining donor cells from the donor; and obtaining donor cells from the donor. In some embodiments, the TLR9 agonist is a CpG-oligonucleotide. In some embodiments the TLR9 agonist is administered systemically.

Also included in the present invention are compositions including a TLR9 agonist and an inhibitor of IDO. In some embodiments, the TLR9 agonist is a CpG-oligonucleotide.

The present invention also includes compositions to induce an immune response, the composition including an antigen, a TLR9 agonist and an inhibitor of IDO. In some embodiments, the TLR9 agonist is a CpG-oligonucleotide.

Unless otherwise specified, “a,” “an,” “the,” and “at least one” are used interchangeably and mean one or more than one.

### BRIEF DESCRIPTION OF THE FIGURES

FIGS. 1A-1D show that CpG-ODNs induce IDO expression exclusively in splenic CD19⁺ DCs. For FIGS. 1A-1C, mice (B6) were injected (50 μg, i.v.) with CpG-ODN (no. 1826; FIGS. 1A and 1C) or control non-CpG-ODN (no. 2138; FIG. 1B). Mice were sacrificed 24 hours later, and spleen tissue sections were stained with anti-IDO antibody and hematoxylin and eosin (H&E) counterstained (FIG. 1A and FIG. 1B) or with anti-IDO antibody and anti-CD19 mAb and stained cells visualized by immunofluorescence (FIG. 1C). In FIG. 1D, splenic DC populations from B6 mice were purified by preparative flow cytometry based on their CD11c and CD19 staining profiles as shown. DCs were incubated with CpG-ODN (no. 1826; 10 μg/ml) for 24 hours and cytospins were stained with a Hoechst nuclei dye and cells stained with anti-IDO Ab were visualized by red immunofluorescence. Original magnifications, X100 (FIGS. 1A-1C); X400 (FIG. 1D).

FIGS. 2A-2D show CD19⁺ DCs acquire potent IDO-dependent, T cell-suppressive functions following CpG-ODN treatment. In FIG. 2A, B6 (wild-type (WT)) and IDO-KO mice were treated with CpG-ODN, and 24 hours later, splenic CD11c⁺ DCs were isolated by AutoMACS and used as stimulators in MLRs with H-2K^{b}-specific responder T cells from BM3 TCR-transgenic mice with (□) or without (▪) addition of IDO inhibitor, 1 mT (100 μM), or excess tryptophan [symbol] In FIGS. 2B-2D, purified DC subsets were isolated by preparative flow cytometry based on CD11c and CD19 (FIG. 2B) and CD11c and 120 G8 (FIGS. 2C and 2D) expression and used as stimulators in MLRs. Mean DC yields were as follows, expressed as percentage of total splenic (CD11c⁺) DCs; CD11c^{high}CD19⁺ (˜10%), CD11c^{high}CD19⁻ (˜40%); CD11c^{low}120 G8⁻ (˜25%), CD11c^{high}120G8⁺ (˜25%). In FIG. 2C, gating criteria for CD11c and 120 G8 staining to purify DC subsets used in MLRs shown in FIG. 2D. FIGS. 2A, 2B, and 2D indicate (percentage) suppression of T cell proliferation due to IDO activity expressed relative to control MLRs containing 1 mT.

FIGS. 3A-3E show IFN-α induces STAT-1 activation and IDO up-regulation in CD19⁺ DCs following CpG-ODN exposure. In FIGS. 3A and 3B, IDO expression in spleen of BALB/c (FIG. 3A) and IFNAR-deficient (FIG. 3B) mice was assessed by immunohistochemical analysis following CpG-ODN (no. 1826) administration (50 μg, i.v.). In FIG. 3C, cell lysates of splenocytes from BALB/c (lanes 1 and 5), IFNAR-KO (lanes 2 and 6), 129/SvJ (lanes 3 and 7), and IFN-γ R-KO (lanes 4 and 8) mice treated with CpG- (lanes 1-4) or non-CpG-ODNs (lanes 4-8) were analyzed by Western blot to detect P-STAT-1 and β-actin. In FIG. 3D, splenocytes from B6 mice treated with CpG-ODNs were stained with CD11c, CD19, and anti-P-STAT-1 mAbs and analyzed by flow cytometry. A two-color plot shows gates used to assess P-STAT-1 expression on gated CD19⁺ and CD19⁻ [symbol] CD11c^{low}; line, CD11c^{high}) DCs in histograms. Bars on histograms indicate P-STAT-1 staining profile (>95% of cells) for DCs from untreated mice. In FIG. 3E, AutoMacs-enriched CD11c⁺ DCs from BALB/c mice were incubated with CpG-ODNs (10 μg/ml) in the absence or in the presence of blocking anti-IFN-γ or anti-IFN-α mAbs as indicated. After five hours, cytospins were stained with Hoechst nuclear dye and anti-P-STAT-1 antibody. Original magnifications, X100 (FIGS. 3A and 3B); X400 (FIG. 3E).

FIGS. 4A and 4B show that CpG oligonucleotides enhance the stimulatory activity of IDO⁺ pDCs from tumor draining lymph nodes (TDLNs). In FIG. 4A, dendritic cells (DC) were isolated by FACS sorting cells from TDLNs of B78H1-GMCSF tumors, based on the following markers: CD11c⁺B220⁺CD19^{NEG} (conventional IDO^{NEG} pDCs), CD11c⁺B220⁺CD19⁺ (IDO⁺ pDCs), and CD11c⁺B220^{NEG} (non-plasmacytoid DCs). These different DC populations were used to stimulate BM3 T cells (TCR-transgenic, recognizing H2K^{b} as an alloantigen on the target DCs). FIG. 4A shows that CpG had a marked effect on the stimulatory ability of CD19⁺ pDCs, increasing their stimulatory activity from a very low level, to a level comparable with the other non-suppressive DCs. FIG. 4B shows that CpG had a lesser effect on the CD19^{NEG} pDCs or on the non-plasmacytoid DCs, both of which were already stimulatory. FIG. 4B further shows that using CpG in combination with 1 MT shows that the degree to which CpG enhanced the stimulatory activity of the CD19⁺ pDCs was markedly increased when IDO was also blocked by 1 MT (open bars).

FIG. 5 shows tumor growth in mice implanted with syngeneic E.G7 tumors (EL4 lymphoma tumors transfected with a chicken ovalbumin transgene) and treated with either the D-isomer of 1 MT (5 mg/day by continuous subcutaneous timed-release pellet) or vehicle control pellets, for 14 days beginning one day after tumor implantation. Subgroups also received either CpG 100 ug i.p. weekly for four doses (arrows) or saline control injections, for a total of four arms as shown in the legend. Each arm contained five mice. Tumor area was measured, and mice were sacrificed when the tumors reached the ethical surrogate endpoint of 300 mm². Data points show averages of remaining mice in each group at the times shown.

### DETAILED DESCRIPTION OF ILLUSTRATIVE EMBODIMENTS OF THE INVENTION

The enzyme indoleamine 2,3-dioxygenase (IDO) has recently been recognized as playing an immunoregulatory role, contributing to peripheral immune tolerance. The present invention is based on the unexpected observation that the systemic administration of a relatively high dose of a Toll-like receptor (TLR) agonist induces the expression of IDO in a subpopulation of dendritic cells (DCs), which then acquire IDO-dependent T cell immunomodulatory functions.

TLRs are a family of pattern recognition receptors that are activated by specific components of microbes and certain host molecules. They constitute the first line of defense against many pathogens and play a crucial role in the function of the innate immune system. TLRs in mammals were first identified in 1997 and it has been estimated that most mammalian species have between ten and fifteen types of Toll-like receptors. Known TLRs include: TLR1 (TLR1 ligands include triacyl lipoproteins); TLR2 (TLR2 ligands include lipoproteins, gram positive peptidoglycan, lipoteichoic acids, fungi, and viral glycoproteins); TLR3 (TLR3 ligands include double-stranded RNA, as found in certain viruses, and poly I:C); TLR4 (TLR4 ligands include lipopolysaccharide and viral glycoproteins); TLR5 (TLR5 ligands include flagellin); TLR6 (TLR6 ligands include diacyl lipoproteins); TLR7 (TLR7 ligands include small synthetic immune modifiers (such as imiquimod, R-848, loxoribine, and bropirimine) and single-stranded RNA); TLR8 (TLR8 ligands include small synthetic compounds and single-stranded RNA); and TLR9 (TLR9 ligands include unmethylated CpG DNA motifs). See, for example, reviews by Akira, “Mammalian Toll-like receptors,” Curr Opin Immunol 2003; 15(1): 5-11 and Akira and Hemmi, “Recognition of pathogen-associated molecular patterns by TLR family,” Immunol Lett 2003; 85(2): 85-95.

With the present invention, an agonist of one or more TLRs may be administered to induce the expression of IDO in a subpopulation of DCs. The terms “agonist” and “agonistic,” as used herein, refer to or describe an agent that is capable of substantially inducing, promoting or enhancing TLR biological activity or TLR receptor activation or signaling. The terms “antagonist” or “antagonistic,” as used herein, refer to or describe an agent that is capable of substantially counteracting, reducing or inhibiting TLR biological activity or TLR receptor activation or signaling.

In some aspects of the present invention, a TLR4 agonist may be administered to induce the expression of IDO. As used herein, a TLR4 agonist refers to an agent that is capable of substantially inducing, promoting or enhancing TLR4 biological activity or TLR4 receptor activation or signaling. Agonists of TLR4 include, but are not limited to, naturally-occurring lipopolysaccharides (LPS), for example, LPS from a wide variety of Gram negative bacteria; derivatives of naturally-occurring LPS; synthetic LPS; bacteria heat shock protein-60 (Hsp60); mannuronic acid polymers; flavolipins; teichuronic acids; S. pneumoniae pneumolysin; bacterial fimbriae, respiratory syncytial virus coat protein; other bacterial pathogen-associated molecular patterns (PAMPs), such as lipoteichoic acid (LTA); and the like.

In a preferred aspect of the present invention, a TLR9 agonist may be administered to induce the expression of IDO. As used herein, a TLR9 agonist refers to an agent that is capable of substantially inducing, promoting or enhancing TLR9 biological activity or TLR9 receptor activation or signaling.

TLR9 is activated by unmethylated CpG-containing sequences, including those found in bacterial DNA or synthetic oligonucleotides (ODNs). Such unmethylated CpG containing sequences are present at high frequency in bacterial DNA, but are rare in mammalian DNA. Thus, unmethylated CpG sequences distinguish microbial DNA from mammalian DNA. See, for example, Janeway and Medzhitov, Ann. Rev. Immunol. 2002; 20: 197; Barton and Medzhitov, Curr. Top. Microbiol. Immunol. 2002; 270:81; Medzhitov, Nat. Rev. Immunol. 2001; 1:135; Heine and Lein, Int. Arch. Allergy Immunol. 2003; 130:180; Modlin, Ann. Allergy Asthma Immunol. 2002; 88:543; and Dunne and O'Neill, Sci. STKE 2003:re3.

A TLR9 agonist may be a preparation of microbial DNA, including, but not limited to, E. coli DNA, endotoxin free E. coli DNA, or endotoxin-free bacterial DNA from E. coli K12. A TLR9 agonist may be isolated from a bacterium, for example, separated from a bacterial source; synthetic, for example, produced by standard methods for chemical synthesis of polynucleotides; produced by standard recombinant methods, then isolated from a bacterial source; or a combination of the foregoing. In many embodiments, a TLR agonist is purified, and is, for example, at least about 70%, at least about 75%, at least about 80%, at least about 85%, at least about 90%, at least about 95%, at least about 98%, at least about 99%, or more, pure.

A TLR9 agonist may be a synthetic oligonucleotide containing unmethylated CpG motifs, also referred to herein as “a CpG-oligodeoxynucleotide,” “CpGODNs,” or “ODN” (see, for example, Hemmi et al. “A Toll-like receptor recognizes bacterial DNA,” Nature 2000; 408: 740-745). At least three types of immunostimulatory CpG-ODNs have been described. Type A (or D) ODNs preferentially activate plasmacytoid dendritic cells (pDC) to produce IFNα, whereas type B (or K) ODNs induce the proliferation of B cells and the secretion of IgM and IL-6. Another type has been generated that combines features of both types A and B termed, and is termed type C. A TLR9 agonist of the present invention may include any of the at least three types of stimulatory ODNs have been described, type A, type B, and type C.

A CpG-oligodeoxynucleotide TLR9 agonist includes a CpG motif. A CpG motif includes two bases to the 5′ and two bases to the 3′ side of the CpG dinucleotide. CpG-oligodeoxynucleotides may be produced by standard methods for chemical synthesis of polynucleotides. CpG-oligodeoxynucleotides may be purchased commercially, for example, from Coley Pharmaceuticals (Wellesley, Mass.), Axxora, LLC (San Diego, Calif.), or InVivogen, (San Diego, Calif.). A CpG-oligodeoxynucleotide TLR9 agonist may includes a wide range of DNA backbones, modifications and substitutions.

In some aspects of the invention, a TLR9 agonist is a nucleic acid that includes the nucleotide sequence 5′CG 3′. In some aspects of the invention, a TLR9 agonist is a nucleic acid that includes the nucleotide sequence 5′-purine-purine-cytosine-guanine-pyrimidine-pyrimidine-3′. In other aspects of the invention, a TLR9 agonist is a nucleic acid that includes the nucleotide sequence 5′-purine-TCG-pyrimidine-pyrimidine-3′. In some aspects of the invention, a TLR9 agonist is a nucleic acid that includes the nucleotide sequence 5′-(TGC)ₙ-3′, where n≧1. In other aspects of the invention, a TLR9 agonist is a nucleic acid that includes the sequence 5′-TCGNN-3′, where N is any nucleotide.

In some aspects, a TLR9 agonist may have a sequence of from about 5 to about 200, from about 10 to about 100, from about 12 to about 50, from about 15 to about 25, from about 5 to about 15, from about 5 to about 10, or from about 5 to about 7 nucleotides in length. In some aspects, a TLR9 agonist may be less than about 15, less than about 12, less than about 10, or less than about 8 nucleotides in length.

A TLR9 agonist of the present invention includes, but is not limited to, any of those described in U.S. Pat. Nos. 6,194,388; 6,207,646; 6,239,116; 6,339,068; and 6,406,705, 6,426,334 and 6,476,000, and published US Patent Applications US 2002/0086295, US 2003/0212028, and US 2004/0248837.

In some aspects, a TLR agonist may be part of a larger nucleotide construct (for example, a plasmid vector, a viral vector, or other such construct). A wide variety of plasmid and viral vector are known in the art, and need not be elaborated upon here. A large number of such vectors has been described in various publications. See, for example, Current Protocols in Molecular Biology, (F. M. Ausubel, et al., Eds. 1987, and updates). Many such vectors are commercially available.

If needed, ODNs containing the same sequence as their stimulatory counterparts, but in which the CpG oligodeoxynucleotides have been replaced by GpC oligodeoxynucleotides, may be used as controls. Such GpC oligodeoxynucleotides do not stimulate TLR9.

A TLR agonist of the present invention may be administered by any suitable means including, but not limited to, for example, oral, rectal, nasal, topical (including transdermal, aerosol, buccal, and sublingual), vaginal, or parenteral (including subcutaneous, intramuscular, intravenous, and intradermal). For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art in light of the present disclosure (see for example, “Remington's Pharmaceutical Sciences” 15th Edition). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. For enteral administration, a TLR agonist may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the form of implants.

In preferred embodiments of the present invention, a TLR agonist is administered systemically. As used herein, systemic delivery is non-local delivery, including any mode of delivery which results in the delivery of a TLR agonist to the systemic circulation before it comes into contact with a lymph node. Systemic delivery includes, for example, intravenous delivery (into a vein), intraarterial delivery (into an artery), intracardiac delivery (into the heart), intraperitoneal delivery, (infusion or injection into the peritoneum), or modes of enteral administration in which the TLR agonist is absorbed into the bloodstream from the gastrointestinal tract.

Therapeutically effective concentrations and amounts may be determined for each application described herein empirically by testing the compounds in known in vitro and in vivo systems, including, but not limited to, any of those described herein, dosages for humans or other animals may then be extrapolated therefrom. Safety profiles have been obtained for the administration of several TLR9 agonists to humans in early clinical trails. These safety profiles have over a more that 1,000-fold dose range, from 0.0025-0.81 milligram (mg) per kilogram (kg). A maximal tolerated dose in humans has not been reported to date. In primates, blood concentrations of about 40 to about 50 micrograms (μg) per milliliter (ml) can be obtained by relatively rapid intravenous administration. See Krieg, “Therapeutic Potential of Toll-like Receptor 9 Activation,” Nature Reviews, 2006; 5:471-484.

With the methods of the present invention, a TLR agonist may be administered at a low dosage. In human subjects, a low dosage of a CpG agonist is about 30 mg or less. A low dosage of a CpG agonist may be about 25 mg or less. A low dosage of a CpG agonist may be about 20 mg or less. A low dosage of a CpG agonist may be about 15 mg or less. A low dosage of a CpG agonist may be about 10 mg or less. A low dosage of a CpG agonist may be about 5 mg or less. A low dosage of a CpG agonist may be about 1 mg or less. A low dosage of a CpG agonist may be about 0.5 mg or less. A low dosage of a CpG agonist may be a range of any of these dosages. For example, a low dosage of a CpG agonist may be from about 0.5 mg to about 30 mg. Such a low dosage may be administered, for example, when a TLR agonist is administered as a vaccine adjuvant. Such a low dosage may, for example, be administered subcutaneously, intradermal, or intratumoral.

With the methods of the present invention, a TLR agonist may be administered at a high dosage. In human subjects a high dosage is greater than 30 mg. A high dosage may, for example, be greater than about 30 mg, greater than about 50 mg, greater than about 75 mg, greater than about 100 mg, greater than about 125 mg, greater than about 150 mg, or more. A high dosage may be up to about 125 mg, up to about 250 mg, up to about 500 mg, or more. Such a high dosage maybe administered, for example, to induce an immunosuppressive effect. Such a low dosage may be administered systemically, including, for example, intravenously.

A TLR agonist may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

A TLR agonist may be formulated as a composition. The compositions of the present invention may be formulated in a variety of forms adapted to the chosen route of administration. The formulations may be conveniently presented in unit dosage form and may be prepared by methods well known in the art of pharmacy. Formulations of the present invention include, for example, pharmaceutical compositions including a TLR9 agonist and a pharmaceutically acceptable carrier. The phrase “pharmaceutically-acceptable” refers to molecular entities and compositions that do not produce an allergic or similar untoward reaction when administered to a human. The preparation of such compositions is well understood in the art. The formulations of this invention may include one or more accessory ingredients including diluents, buffers, binders, disintegrants, surface active agents, thickeners, lubricants, preservatives (including antioxidants), and the like.

In preferred embodiments of the methods of the present invention, a TLR agonist is administered in an amount effective to induce the expression of IDO in a subpopulation of DCs. A TLR agonist may be administered in an amount effective to induce the expression of IDO in a IDO-competent subset of DCs. A TLR agonist may be administered in an amount effective to induce the expression of IDO in a subpopulation of splenic DCs. The subpopulation of splenic DC cells may be defined by any of various functional characteristics and/or phenotypic markers. A TLR agonist may be administered in an amount effective to induce the expression of IDO in a subpopulation of plasmacytoid DCs, including those in peripheral circulation, a lymph node, and/or bone marrow.

The IDO enzyme is well characterized (see, for example, Taylor et al., FASEB Journal 1991; 5:2516-2522; Lee et al., Laboratory Investigation, 2003; 83:1457-1466; and Grohmann et al., Trends in Immunology 2003; 24:242-248) and compounds that serve as substrates or inhibitors of the IDO enzyme are known. For example, Southan (Southan et al, Med. Chem. Res., 1996; 343-352) utilized an in vitro assay system to identify tryptophan analogues that serve as either substrates or inhibitors of human IDO. Methods for detecting the expression of IDO in cells are well known and include, but are not limited to, any of those described herein.

With the present invention, a TLR agonist may be administered in an amount effective to induce the expression of IDO in mouse CD19⁺ splenic DCs and the equivalent subpopulation of human splenic DCs. With the present invention, a TLR agonist may be administered in an amount effective to induce the expression of IDO in mouse CD11c⁺ CD19⁺ splenic DCs and the equivalent subpopulation in human splenic DCs (see Example 1 and Mellor et al., J. Immunol., “Cutting Edge: CpG Oligonucleotides Induce Splenic CD19+ Dendritic Cells to Acquire Potent Indoleamine 2,3-Dioxygenase-Dependent T Cell Regulatory Functions via IFN Type I Signaling” 2005; 175(9):5601-5).

With the present invention, a TLR agonist may be administered in an amount effective to induce the expression of IDO in human CD11c⁺, CD123⁺ splenic DCs. With the present invention, a TLR agonist may be administered in an amount effective to induce the expression of IDO in CD11c⁺, CCR6⁺ human splenic DCs.

With the present invention, a TLR agonist may be administered in an amount effective to induce the expression of IDO in a subpopulation of dendritic cells in which B7 ligation induces activation of the “transcription factor signal transducer and activator of transcription-1” (STAT1). The identification of this subpopulation of DC cells is described, for example, in Baban et al., International Immunol., “A Minor Population of Splenic Dendritic Cells Expression CD19 Mediates IDO-Dependent T Cell Suppression Via Type I IFN Signaling Following B7 Ligation” 2005; 17(7):909-919. Signal Transducers and Activators of Transcription (STATs) are a family of cytoplasmic proteins with roles as signal messengers and transcription factors that participate in normal cellular responses to cytokines and growth factors (Turkson and Jove, “STAT proteins: novel molecular targets for cancer drug discovery,” Oncogene 2000; 19:6613-26). STAT1 (signal transducer and activator of transcription #1) is an 88 kDa member of the STAT family of cytoplasmic transcription factors. STAT members generally mediate cytokine, growth factor and hormone receptor signal transduction. STAT1 is associated with type I and II interferon signaling.

With the present invention, a TLR agonist may be administered in an amount effective to induce Type I-IFN-dependent IDO expression in a subpopulation of dendritic cells, including, but not limited to a subpopulation of splenic dendritic cells. With the present invention, a TLR agonist may be administered in an amount effective to induce IDO expression in a subpopulation of dendritic cells, including, but not limited to a subpopulation of splenic dendritic cells, in which induction of STAT1 is regulated by Type I interferons.

The present invention includes methods of suppressing T cell activation to an antigen in a subject by administering a TLR agonist and the antigen to the subject, resulting in the suppression of an immune response to the antigen. The present invention includes methods of inducing tolerance to an antigen in a subject by administering a TLR agonist and the antigen to the subject. In preferred embodiments, the TLR agonist is a TLR9 agonist. The TLR agonist may be administered in an amount effective to induce IDO expression in splenic DC. The TLR agonist may be administered before, coincident to, and/or after the administration of the antigen. An antigen can include, for example, a vaccine preparation or an alloantigen. A vaccine may include one or more immunogenic peptides. The vaccine may include genetically modified cells.

The present invention includes methods of method of preventing allograft rejection in a subject by administering to the subject a TLR agonist and one or more alloantigens present in the allograft. In preferred embodiments, the TLR agonist is a TLR9 agonist. The TLR agonist may be administered in an amount effective to induce IDO expression in a subpopulation of splenic DCs. The TLR agonist may be administered before, coincident to, and/or after the administration of the one or more alloantigen.

The present invention includes methods of preventing allograft rejection in a recipient by administering a TLR agonist to the recipient after the transplantation of the allograft into the recipient. In preferred embodiments, the TLR agonist is a TLR9 agonist. The TLR agonist may be administered in an amount effective to induce IDO expression in a subpopulation of splenic DCs.

As an example of the prevention of allograft rejection, the role of IDO in the placenta is to suppress T cell responses against the genetically foreign fetus. If IDO activity were inhibited in the placenta, the fetus would be unable to protect itself against maternal T cells and would be rejected. Inhibition of IDO with 1-methyltryptophan results in prompt, T cell-mediated rejection of an allogeneic fetus. There is no effect of the inhibitor on genetically identical (syngeneic) control fetuses, demonstrating that the inhibitor itself was not toxic. Thus, expression of IDO serves as a marker of suppression of T cell activation, and plays a significant role in allogeneic pregnancy and therefore other types of transplantation. The administration of an inhibitor of IDO, 1-methyl-tryptophan, induces specific and uniform rejection of allogeneic conceptus. See U.S. Pat. Nos. 6,451,840 and 6,482,416. Rejection is T cell driven since a single paternally-inherited fetal MHC class I alloantigen provokes embryo loss, and rejection does not occur if maternal lymphocytes are absent when IDO activity is inhibited or the mother does not have functional T cells.

In addition to a role in pregnancy, IDO-expressing cells are widely distributed in primary and secondary lymphoid organs. Monocytes that have differentiated under the influence of macrophage colony-stimulating factor acquire the ability to suppress T cell proliferation in vitro via rapid and selective degradation of tryptophan by IDO. In Munn et al. (J Exp Med 1999; 189:1363-72), IDO was induced in macrophages by a synergistic combination of the T cell-derived signals IFN-gamma and CD40-ligand. Inhibition of IDO with the 1-methyl analogue of tryptophan prevented macrophage-mediated suppression. Purified T cells activated under tryptophan-deficient conditions were able to synthesize protein, enter the cell cycle, and progress normally through the initial stages of G1, including up regulation of IL-2 receptor and synthesis of IL-2. However, in the absence of tryptophan, cell cycle progression halted at a mid-G1 arrest point. Restoration of tryptophan to arrested cells was not sufficient to allow further cell cycle progression nor was costimulation via CD28. T cells could exit the arrested state only if a second round of T cell receptor signaling was provided in the presence of tryptophan. Antigen-presenting cells can regulate T cell activation via tryptophan catabolism and the expression of IDO by certain antigen presenting cells in vivo allows them to suppress unwanted T cell responses.

The present invention also includes methods of preventing graft versus host disease (GVHD) in a recipient, the method including administering to the donor a TLR agonist and one or more alloantigens present in the recipient, wherein the TLR9 agonist and the one or more alloantigens present in the recipient are administered to the donor prior to obtaining donor cells from the donor; obtaining donor cells from the donor; and administering the donor cells to the recipient. In preferred embodiments, the TLR agonist is a TLR9 agonist. GVHD is a complication of an allogeneic bone marrow or cord blood transplant (BMT) in which functional immune cells in the transplanted marrow recognize the recipient as “foreign” and mount an immunologic attack. Thus, GVHD is a pathological condition in which cells from the transplanted tissue of a donor initiate an immunologic attack on the cells and tissue of the recipient. After bone marrow transplantation, T cells present in the graft, either as contaminants or intentionally introduced into the host, attack the tissues of the transplant recipient after perceiving host tissues as antigenically foreign. A wide range of host antigens, also referred to herein as “alloantigens” can initiate GVHD, among them the HLAs. However, graft-versus-host disease can occur even when HLA-identical siblings are the donors. HLA-identical siblings or HLA-identical unrelated donors (called a minor mismatch as opposed to differences in the HLA antigens, which constitute a major mismatch) often still have genetically different proteins that can be presented on the MHC.

The present invention includes methods of preconditioning a recipient of an allograft to suppress allograft rejection in the recipient, the method including administering to the recipient a TLR agonist and one or more alloantigens present in the allograft, wherein the TLR agonist and the one or more alloantigens present in the allograft are administered to the recipient prior to allografting; and transplanting the allograft into the recipient. In preferred embodiments, the TLR agonist is a TLR9 agonist. The TLR agonist may be administered in an amount effective to induce IDO expression in splenic DC.

The present invention includes methods of enhancing an immune response in a subject by administering to the subject a TLR agonist and an inhibitor of IDO. In preferred embodiments, the TLR agonist is a TLR9 agonist. The administration of a TLR agonist may take place before, during, and/or after the administration of an inhibitor of IDO. IDO inhibitors include, but are not limited to, 1-methyl-tryptophan, β-(3 benzofuranyl)-alanine, β-[3-benzo(b)thienyl]-alanine, 6-nitro-tryptophan, and derivatives thereof. An inhibitor of indoleamine-2,3-dioxygenase may be a L isomer of an inhibitor of indoleamine-2,3-dioxygenase, a D isomer of an inhibitor of indoleamine-2,3-dioxygenase, or a racemic mixture of an inhibitor of indoleamine-2,3-dioxygenase. In some embodiments, a preferred IDO inhibitor is 1-methyl-tryptophan, also referred to as 1 MT or 1M-T. See, for example, published U.S. Patent Application Nos. 2004/0234623 and 2005/0186289. Additional examples of compounds that inhibit IDO activity are brassinin derivatives described by Gaspari et al. (J Medicinal Chem 2006; 49(2):684-92), a series of indole derivatives described in patent application PCT/US04/05154, and a series of compounds derived from naphtoquinones described in WO/2006/005185. Inhibitors of the IDO enzyme are readily commercially available, for example, from Sigma-Aldrich Chemicals, St. Louis, Mo.

The present invention includes methods of enhancing an immune response to an antigen in a subject by administering to the subject both a TLR agonist and an IDO inhibitor. Antigens to which the administration of a combination of a TLR agonist and an IDO inhibitor will result in an enhanced immune response include, but are not limited to viral antigens, tumor antigens, and bacterial antigens. In preferred embodiments, the TLR agonist is a TLR9 agonist. The TLR agonist may be administered in an amount effective to induce IDO expression in an IDO competent subset of DC.

The stimulation of an immune response may be measured by any of many standard methods well known in the immunological arts. As used herein, a mixed leukocyte response (MLR) is a well-known immunological procedure, for example, as described in the examples herein. As used herein, T cell activation by an antigen-presenting cell is measured by standard methods well known in the immunological arts. As used herein, a reversal or decrease in the immunosuppressed state in a subject is as determined by established clinical standards. As used herein, the improved treatment of an infection is as determined by established clinical standards. The determination of immunosuppression mediated by an antigen presenting cell expressing indoleamine-2,3-dioxygenase (IDO) includes the various methods as described in the examples herein.

Certain pathological conditions, such as parasitic infections, AIDS (caused by the human immunodeficiency virus (HIV)) and latent cytomegaloviral (CMV) infections, are extremely difficult to treat since the macrophages act as reservoirs for the infectious agent. Even though the cells are infected with by a foreign pathogen, they are not recognized as foreign. The methods of the present invention may be used to treat such pathological conditions including, but not limited to, viral infections, infection with an intracellular parasite, and infection with an intracellular bacteria. Viral infections treated include, but are not limited to, infections with the human immunodeficiency virus (HIV) or cytomegalovirus (CMV). Intracellular bacterial infections treated include, but are not limited to infections with Mycobacterium leprae, Mycobacterium tuberculosis, Listeria monocytogenes, and Toxplasma gondii. Intracellular parasitic infections treated include, but are not limited to, Leishmania donovani, Leishmania tropica, Leishmania major, Leishmania aethiopica, Leishmania mexicana, Plasmodium falciparum, Plasmodium vivax, Plasmodium ovale, and Plasmodium malariae.

The efficacy of treatment of an infection may be assessed by any of various parameters well known in the art. This includes, but is not limited to, a decrease in viral load, an increase in CD4⁺ T cell count, a decrease in opportunistic infections, eradication of chronic infection, and/or increased survival time.

The present invention includes methods of stimulating CD8 T cell activation in a subject by administering a TLR agonist and an inhibitor of IDO to the subject. In preferred embodiments, the TLR agonist is a TLR9 agonist.

Current experimental methods of cancer treatment include tumor vaccination protocols consisting in the administration of tumor peptides or whole cell tumor vaccines with CpG ODNs as immunostimulatory adjuvants. Currently CpG ODNs have been utilized as an adjuvant along with a tumor vaccine. However, as shown by the present invention, the administration of a CpG ODN adjuvant can induce the expression of IDO in a subpopulation of DCs that may lead to partial or full immunosuppression, precluding the full immunostimulatory capacity of DCs and therefore potentially dampening the immune response to tumor specific antigens. The present invention provides methods to enhance the immunostimulatory capacity of DCs to tumor antigens by co-administration of one or more inhibitors of IDO along with the administration of a TLR agonist. Therefore, the present invention includes methods of treating cancer in a subject by administering to the subject a TLR agonist, an inhibitor of IDO, and a tumor vaccine.

The present invention includes methods of treating cancer in a subject by administering to the subject a TLR agonist and an inhibitor of IDO. The present invention also includes methods of treating cancer in a subject by administering to the subject a TLR agonist, an inhibitor of IDO, and one or more additional therapeutic agents. In preferred embodiments, the TLR agonist is a TLR9 agonist. Additional therapeutic treatments include, but are not limited to, surgical resection, radiation therapy, chemotherapy, hormone therapy, anti-tumor vaccines, antibody based therapies, whole body irradiation, bone marrow transplantation, peripheral blood stem cell transplantation, and the administration of chemotherapeutic agents (also referred to herein as “antineoplastic chemotherapy agent”). Antineoplastic chemotherapy agents include, but are not limited to, cyclophosphamide, methotrexate, 5-fluorouracil, doxorubicin, vincristine, ifosfamide, cisplatin, gemcitabine, busulfan (also known as 1,4-butanediol dimethanesulfonate or BU), ara-C (also known as 1-beta-D-arabinofuranosylcytosine or cytarabine), adriamycin, mitomycin, cytoxan, methotrexate, and combinations thereof. The administration of a TLR agonist may take place before, during, and/or after the administration of an additional chemotherapeutic agent. Additional therapeutic agents include, for example, one or more cytokines, an antibiotic, antimicrobial agents, antiviral agents, such as AZT, ddI or ddC, and combinations thereof. The cytokines used include, but are not limited to, IL-1α, IL-1β, IL-2, IL-3, IL-4, IL-6, IL-8, IL-9, IL-10, IL-12, IL-18, IL-19, IL-20, IFN-α, IFN-β, IFN-γ, tumor necrosis factor (TNF), transforming growth factor-β (TGF-β), granulocyte colony stimulating factor (G-CSF), macrophage colony stimulating factor (M-CSF), granulocyte-macrophage colony stimulating factor (GM-CSF)) (U.S. Pat. Nos. 5,478,556, 5,837,231, and 5,861,159), or Flt-3 ligand (Shurin et al., Cell Immunol. 1997; 179:174-184). Antitumor vaccines include, but are not limited to, peptide vaccines, whole cell vaccines, genetically modified whole cell vaccines, recombinant protein vaccines or vaccines based on expression of tumor associated antigens by recombinant viral vectors.

The efficacy of the administration of a TLR agonist; the administration of a TLR agonist along with an antigen; the administration of a TLR agonist along with an inhibitor of IDO; the administration of a TLR agonist along with an antigen and an IDO inhibitor; the administration of a TLR agonist along with an IDO inhibitor and an additional therapeutic agent; or the administration of a TLR agonist along with an antigen, an IDO inhibitor, and an additional therapeutic agent may be assessed by any of a variety of parameters well known in the art. This includes, for example, determinations of an increase in the delayed type hypersensitivity reaction to tumor antigen, determinations of a delay in the time to relapse of the post-treatment malignancy, determinations of an increase in relapse-free survival time, determinations of an increase in post-treatment survival, determination of tumor size, determination of the number of reactive T cells that are activated upon exposure to the vaccinating antigens by a number of methods including ELISPOT, FACS analysis, cytokine release, or T cell proliferation assays.

The tumors to be treated by the present invention include, but are not limited to, melanoma, colon cancer, pancreatic cancer, breast cancer, prostate cancer, lung cancer, leukemia, lymphoma, sarcoma, ovarian cancer, Kaposi's sarcoma, Hodgkin's Disease, Non-Hodgkin's Lymphoma, multiple myeloma, neuroblastoma, rhabdomyosarcoma, primary thrombocytosis, primary macroglobulinemia, small-cell lung tumors, primary brain tumors, stomach cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. As used herein, “tumor” refers to all types of cancers, neoplasms, or malignant tumors found in mammals.

The efficacy of treatment of a tumor may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

The present invention includes isolated cell populations preconditioned to minimize graft versus host disease when transplanted into a donor recipient. The cell populations may be obtained by administering to the donor a TLR9 agonist and one or more alloantigens present in the recipient, wherein the TLR9 agonist and the one or more alloantigens present in the recipient are administered to the donor prior to obtaining donor cells from the donor; and obtaining donor cells from the donor. The TLR agonist may be administered in an amount effective to induce IDO expression in an IDO-competent subset of DCs. The TLR agonist may be administered in an amount effective to induce IDO expression in subpopulation of splenic DCs. Such preconditioned cell populations can be used in a number of immunotherapies, including, for example, for the prevention of GVHD, to decrease the likelihood of rejection of an allograft or xenotransplanted tissue or organ, or the treatment of autoimmune diseases.

As used herein, the term “subject” includes, but is not limited to, humans and non-human vertebrates. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits. As used herein, the terms “subject,” “individual,” “patient,” and “host” are used interchangeably. In preferred embodiments, a subject is a mammal, particularly a human.

As used herein “in vitro” is in cell culture and “in vivo” is within the body of a subject.

As used herein, “treatment” or “treating” include both therapeutic and prophylactic treatment. To treat a disease or condition shall mean to intervene in such disease or condition so as to prevent or slow the development of, prevent or slow the progression of, halt the progression of, or eliminate the disease or condition.

As used herein, the term “pharmaceutically acceptable carrier” refers to one or more compatible solid or liquid filler, diluents or encapsulating substances which are suitable for administration to a human or other vertebrate animal.

As used herein, the term “isolated” as used to describe a compound shall mean removed from the natural environment in which the compound occurs in nature. In one embodiment isolated means removed from non-nucleic acid molecules of a cell.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In some embodiments, an “effective amount” of a TLR agonist is an amount that results in a reduction of at least one pathological parameter. Thus, for example, in some aspects of the present invention, an effective amount of a TLR agonist is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the TLR agonist.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### CpG Oligonucleotides Induce Splenic CD19⁺ Dendritic Cells to Acquire Potent Indoleamine 2,3-Dioxygenase-Dependent T Cell Regulatory Functions Via IFN Type 1 Signaling

CpG oligodeoxynucleotides (CpG-ODNs) stimulate innate and adaptive immunity by binding to TLR9 molecules. Paradoxically, expression of the immunoregulatory enzyme indoleamine 2,3-dioxygenase (IDO) is induced following intravenous (i.v.) CpG-ODN administration to mice. CpG-ODNs induced selective IDO expression by a minor population of splenic CD19⁺ dendritic cells (DCs) that did not express the plasmacytoid DC marker 120 G8. Following systemic CpG-ODN treatment, CD19⁺ DCs acquired potent IDO-dependent T cell suppressive functions in a dominant fashion. Signaling through IFN type I receptors was essential for IDO up-regulation, and CpG-ODNs induced selective activation of STAT-1 in CD19⁺ DCs. Thus, CpG-ODNs delivered systemically at relatively high doses elicited potent T cell regulatory responses by acting on a discrete, minor population of splenic DCs. The ability of CpG-ODNs to induce both stimulatory and regulatory responses offers novel opportunities for using them as immunomodulatory reagents but may complicate therapeutic use of CpG-ODNs to stimulate antitumor immunity in cancer patients.

Recently, a minor population of murine splenic DCs expressing CD19 was described that responded to CTLA4-Ig-mediated ligation of CD80/86 (B7) molecules by expressing the intracellular enzyme indoleamine 2,3-dioxygenase (IDO) (Baban et al., “A minor population of splenic dendritic cells expressing CD19 mediates IDO-dependent T cell suppression via type I IFN signaling following B7 ligation,” Int. Immunol. 2005; 17: 909-919). IDO catalyzes the first and rate limiting step of oxidative tryptophan catabolism, and functional IDO expression is linked mechanistically to suppression of T cell-mediated immunity in multiple systems (Mellor and Munn, “IDO expression in dendritic cells: tolerance and tryptophan catabolism,” Nat. Rev. Immunol. 2004; 4: 762-774). Following B7 ligation, splenic DCs acquired potent IDO-dependent regulatory functions that prevented proliferation of alloantigen-specific T cells in vitro and in vivo (Mellor et al., “Cutting edge: induced indoleamine 2,3-dioxygenase expression in dendritic cell subsets suppresses T cell clonal expansion,” J. Immunol. 2003; 171:1652-1655; and Mellor et al., “Specific subsets of murine dendritic cells acquire potent T cell regulatory functions following CTLA4-mediated induction of indoleamine 2,3-dioxygenase,” Int. Immunol. 2004; 16:1391-1401). This response was highly selective as IDO up-regulation occurred exclusively in minor DC populations expressing B220, CD8α, and CD19 following B7 ligation. However, acquired T cell regulatory functions of CD19⁺ DCs were potent and dominant because the underlying T cell stimulatory functions of the majority of splenic (IDO⁻) DCs were not evident until they were separated from IDO⁺ DCs. In the current study, the effects of CpG-ODN mediated ligation of TLR9 on the T cell stimulatory functions of splenic DCs were evaluated. It was found that systemic administration of CpG-ODNs induced selective IFN-α-dependent activation of STAT-1 and IDO up-regulation in splenic CD19⁺ DCs, which acquired potent IDO-dependent T cell regulatory functions as a consequence.

### Materials and Methods

Mice. All mice were bred in a pathogen-free facility. BM3 TCR transgenic mice, IDO-deficient (IDO-knockout (KO)), and IFN-type I receptor (IFNAR)-deficient (IFNAR-KO) mice were as previously described by Baban et al. (Baban et al., Int. Immunol. 2005; 17: 909-919; Mellor et al., J. Immunol. 2003; 171:1652-1655; and Baban et al., “Indoleamine 2,3-dioxygenase expression is restricted to fetal trophoblast giant cells during murine gestation and is maternal genome specific,” J. Reprod. Immunol. 2004; 61:67-77). All procedures involving mice were reviewed and approved by the local Institutional Animal Care and Use Committee.

Antibodies and immunohistochemistry. Details of antibodies (Abs) and protocols used to detect IDO, CD11c, CD19, IFN-α, IFN-γ, and phospho-STAT-1 (P-STAT-1) by immunohistochemical, immunofluorescence, and cytospin staining were as described previously (Baban et al., Int. Immunol. 2005; 17:909-919; Mellor et al., Int. Immunol. 2004; 16:1391-1401; and Baban et al., J. Reprod. Immunol. 2004; 61:67-77). 120 G8 mAb (Asselin-Paturel et al., “Mouse strain differences in plasmacytoid dendritic cell frequency and function revealed by a novel monoclonal antibody,” J. Immunol. 2003; 171:6466-6477) was supplied by Schering-Plough with kind permission from Drs. G. Trinchieri (National Institute of Allergy and Infectious Diseases, National Institutes of Health) and C. Asselin-Paturel (Dardilly, France) and was biotinylated using EZ-Link NHS-LC-Biotin (catalog no. 21336; Pierce).

CpG-ODNs. CpG-ODNs with fully phosphorothioate backbones were purchased from Coley Pharmaceuticals. CpG-B (catalog no. 1826) was TCCATGACGTTCCTGACGTT (SEQ ID NO:1); control non-CpG-B (catalog no. 2138) was TCCATGAGCTTCCTGAGCTT (SEQ ID NO:2) For in vivo treatment, mice were injected i.v. with relatively high doses of ODN (50 μg/mouse); in previous reports, doses up to 20 μg/mouse were used to induce IDO in lung (Hayashi et al., “Inhibition of experimental asthma by indoleamine 2,3-dioxygenase,” J. Clin. Invest. 2004; 114:270-279). For in vitro treatment, DCs were incubated with CpG-ODNs at 12.5 μg/ml.

Preparation of DCs and MLRs. Procedures for isolating DCs and DC subsets from spleen and evaluating their T cell stimulatory functions in vitro using responder T cells from BM3 TCR transgenic mice were as described previously (Baban et al., Int. Immunol. 2005; 17:909-919; and Mellor et al., Int. Immunol. 2004; 16:1391-1401). In brief, freshly isolated spleens were injected with collagenase and homogenous cell suspensions were prepared by disrupting tissues. Cells were stained with Abs and AutoMacs (CD11c⁺; >80% enriched) or preparative flow cytometric methods (>98% pure) were used to select specific DC populations. DCs were mixed with BM3 responder T cells and T cell proliferation assessed after 72 hours in a thymidine incorporation assay.

Preparative and analytical flow cytometry. Preparative cell sorts were performed on cells stained with fluorochrome-conjugated monoclonal antibodies (mAbs) (sources as detailed above) using a Mo-Flo 4 way flow cytometer (DakoCytomation) equipped with 488 nanometer (nm) argon (for FITC, PE, PE-CY5) and 647 nm krypton (for allophycocyanin) lasers. Cells were gated based on forward and side scatter properties and on marker combinations to select cells of interest. Analytical flow cytometry to detect intracellular P-STAT-1 was performed by staining splenocytes from CpG-ODN-treated mice with CD11c and CD19 mAbs before fixing cells and staining with rabbit anti-mouse P-STAT-1α (Tyr⁷⁰¹) antibody using the manufacturer's protocol.

Western blot analysis to detect P-STAT-1. Cell lysates from freshly isolated splenocytes were gel electrophoresed and stained to detect P-STAT-1α and β-actin using antibody manufacturer's protocols, as described by Fallarino et al. (Fallarino et al., “Functional expression of indoleamine 2,3-dioxygenase by murine CD8⁺ dendritic cells,” Int. Immunol. 2002; 14:65-68).

### Results and Discussion

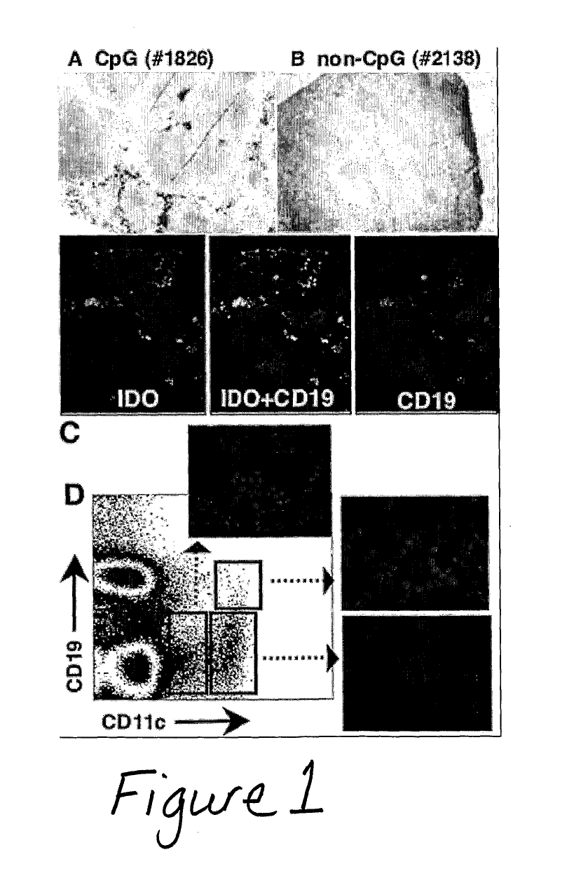

CpG-ODNs induce selective IDO up-regulation in splenic CD19⁺ DCs. B6 mice were injected i.v. with relatively high doses (50 μg/mouse) of CpG-ODN (no. 1826; CpG-B) or non-CpG-ODN (no. 2138) with a near-identical DNA sequence containing no CpG motifs, and IDO expression was assessed 24 hours later by immunohistochemical analysis of spleen. In CpG-ODN-treated mice, IDO⁺ cells were dispersed throughout splenic red pulp, but not in lymphoid follicles (FIG. 1A), and displayed a distinctive plasmacytoid-like morphology. Similar patterns of IDO expression were observed in mice exposed to soluble CTLA4 (CTLA4-Ig), which ligates B7 molecules (Baban et al., Int. Immunol. 2005; 17:909-919; Mellor et al., Int. Immunol. 2004; 16:1391-1401).

As in these previous studies, few IDO⁺ cells were present in spleen of untreated mice, and treatment with non-CpG-ODN did not increase the number of IDO⁺ cells (FIG. 1B). IDO up regulation in spleen was not detected when mice were treated with lower amounts of CpG-ODNs injected i.v. (no. 1826, ≦40 μg) or when CpG-ODNs (no. 1826; 50 μg) were injected into the peritoneum. Two-color immunofluorescence staining revealed that many IDO⁺ cells coexpressed CD19 (FIG. 1C). In contrast, CD19⁺ cells located in lymphoid follicles (i.e., B cells) did not coexpress IDO. No increases in numbers of IDO⁺ cells were observed in other lymphoid tissues from mice exposed to CpG-ODNs.

To further characterize IDO⁺ cells, splenic DC populations were purified from untreated B6 mice by rapid preparative flow cytometry (Mo-Flo, >98% pure) using gating criteria (FIG. 1D) for CD11c and CD19 expression, as previously described (see Baban et al., Int. Immunol. 2005; 17:909-919; and Mellor et al., Int. Immunol. 2004; 16:1391-1401). Purified DCs were incubated with CpG-ODN no. 1826 (10 μg/ml), and 24 hours later, cytospins were stained with anti-IDO antibody. Most (>90%) purified CD11c^{high}CD19⁺ DCs expressed IDO, while few (<5%) cells expressed IDO in purified CD11c^{high}CD19⁻ and CD11c^{low} DC populations (FIG. 1D). Non-CpG-ODN (no. 2138) did not induce IDO expression in any purified DC subset. Collectively, these data revealed that CpG-ODNs induced selective IDO up-regulation in a minor subset of CD19⁺ DCs located in splenic red pulp.

IDO up-regulation in DCs following CpG-ODN administration appears paradoxical because CpG-ODNs have potent immunostimulatory properties (Klinman, “Immunotherapeutic uses of CpG oligodeoxynucleotides,” Nat. Rev. Immunol. 2004; 4:249-258), and induced IDO expression correlates with potent inhibition of T cell-mediated adaptive immunity in experimental models of tumor growth, tissue transplantation, autoimmune disease, and pregnancy (Mellor and Munn, Nat. Rev. Immunol. 2004; 4:762-774; and Grohmann et al., “Tolerance, DCs and tryptophan: much ado about IDO,” Trends Immunol. 2003; 24:242-248). One potential resolution of this paradox is that high doses of CpG-ODNs (50 μg/mouse) administered i.v. were needed to up-regulate IDO in spleen. It has recently been reported that CpG-ODNs suppressed symptoms of asthma in an experimental mouse model due to induction of IDO (Hayashi et al., J. Clin. Invest. 2004; 114:270-279). In this study, 20 μg/mouse of CpG-ODNs were injected i.v., which induced IDO in lung DCs and epithelial cells. However, this lower dosing regimen did not induce IDO activity in spleen. These findings were consistent with these outcomes because 50 μg/mouse of CpG-ODN no. 1826 was the minimum dose required to up-regulate IDO in spleen. It is unclear why injection of 50 μg/mouse of CpG-ODN into the peritoneum did not induce IDO in spleen; presumably, this is due to different pharmacokinetics of CpG-ODNs administered directly into blood or into the peritoneum. Highly selective IDO induction in splenic CD19⁺ DC populations was also observed in previous studies using CTLA4-Ig as a B7 ligand (Baban et al., Int. Immunol. 2005; 17:909-919; and Mellor et al., Int. Immunol. 2004; 16:1391-1401).

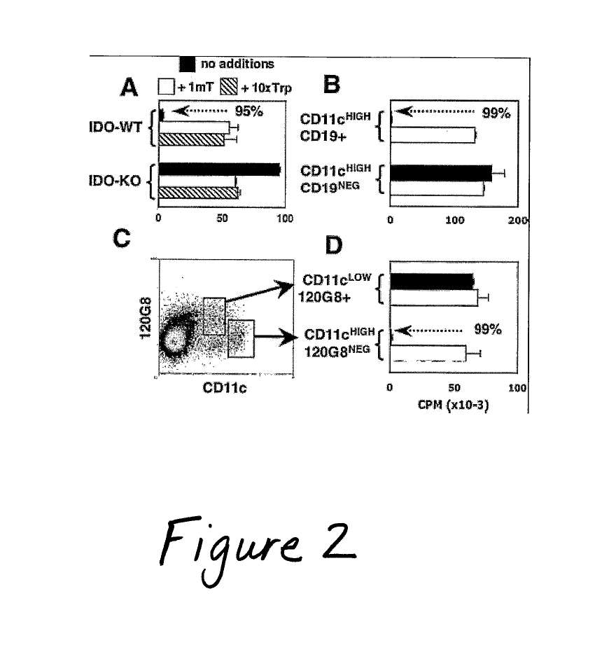

CpG-ODNs induce CD19⁺ DCs to mediate IDO-dependent T cell suppression. To test whether CpG-ODN (i.v.) administration affected T cell stimulatory functions of splenic DCs, specific DC populations were purified from spleen of CpG-ODN-treated mice and their ability to stimulate T cell proliferation in vitro was assessed, using methods previously described (see Baban et al., Int. Immunol. 2005; 17:909-919; and Mellor et al., Int. Immunol. 2004; 16:1391-1401). Splenic CD11c⁺ (AutoMacs enriched) DCs from B6 (wild-type) mice exposed to CpG-ODNs (no. 1826) did not stimulate T cell proliferation (FIG. 2A). However, addition of IDO inhibitor 1-methyl-D-tryptophan (1 mT; 100 μM) or excess tryptophan (10X Trp; 250 μM final) to MLRs restored potent T cell stimulatory functions DCs from CpG-ODN-treated mice. In contrast, DCs from IDO-KO (B6) mice treated with CpG-ODNs stimulated robust T cell responses that were not enhanced by addition of 1 mT or excess tryptophan. This indicates that the immunosuppressive capacity of the CD11c+ CD19+ DCs cells obtained after systemic administration of a high dose of CpG ODNs was dominant over the immunostimulatory capacity of the CD19− DCs (FIG. 2A) as evidenced by the fact that total population of CD11c+ DCs was unable to stimulate proliferation of alloreactive T cells in a MLR culture. It also demonstrates that the immunosuppressive capacity of this subpopulation of splenic DCs is mediated by IDO and that administration of IDO inhibitors can reverse the immunosuppressive effect.

Consistent with previous studies on DCs from CTLA4-Igtreated mice (Baban et al., Int. Immunol. 2005; 17:909-919; and Mellor et al., Int. Immunol. 2004; 16:1391-1401), preliminary studies revealed that purified CD11c^{high}, but not CD11c^{low}, DCs from CpG-ODN-treated mice mediated IDO-dependent T cell suppression. Therefore, it was tested whether purified populations of splenic DCs expressing CD19 and plasmacytoid DCs (pDCs) expressing the marker 120 G8 mediated IDO-dependent T cell suppression following CpG-ODN administration. CD11c^{high}CD19⁺ DCs and CD11c^{high}CD19⁻ DCs were purified from CpG-ODN-treated B6 mice using gating criteria as previously described (Baban et al., Int. Immunol. 2005; 17:909-919). Purified CD11c^{high}CD19⁺ DCs did not stimulate T cell proliferation, unless 1 mT was added, while CD11c^{high}CD19⁻ DCs stimulated robust T cell proliferation, which was not enhanced in the presence of 1 mT (FIG. 2B). In separate experiments, pDCs expressing 120 G8 and relatively low levels of CD11c (CD11c^{low}120 G8⁺) were purified and their ability to stimulate T cell proliferation compared with copurified non-pDCs, defined as CD11c^{high}120 G8⁻ (FIG. 2C). CD11c^{high}120 G8⁻ DCs from CpG-ODN-treated mice did not stimulate T cell proliferation, unless 1 mT was added to MLRs, which restored robust T cell stimulatory functions (FIG. 2D).

In contrast, copurified pDCs (CD11c^{low}120 G8⁺) stimulated robust T cell responses, which were not further enhanced by adding 1 mT. In each case, purified DC populations that suppressed T cell proliferation following in vivo CpG-ODN exposure were minor DC subsets (˜15-25% of total splenic CD11c⁺ DCs). However, as in previous studies with DCs from CTLA4-Ig-treated mice (Baban et al., Int. Immunol. 2005; 17:909-919; Mellor et al., Int. Immunol. 2004; 16:1391-1401), T cell regulatory functions of these minor DC populations predominated over the T cell stimulatory functions of the majority of splenic DCs (e.g., CD11c^{high}CD19⁻ DCs and pDCs), which manifested only when they were separated from DCs that mediated IDO-dependent T cell suppression (see, for example, FIG. 2). Thus, IDO-dependent suppression was mediated exclusively by a discrete, minor population of splenic CD19⁺ DCs that could be distinguished from pDCs expressing 120 G8 following systemic exposure to CpG-ODNs.

A recent study reported that splenic pDCs, defined as CD11c⁺ B220⁺ 120 G8⁺, suppressed T cell mediated delayed-type hypersensitivity responses following CD200R ligation due to selective IDO induction in this DC subset (Fallarino et al., J. Immunol. 2004; 173:3748-3754). In the current study, systemic CpG-ODN exposure did not inhibit the robust T cell stimulatory functions of pDCs, defined as CD11c^{low}120 G8⁺. The distinct outcomes from the two studies might arise for several reasons, including use of different mouse strains, different ligands to induce IDO, which may act on distinct DC populations ((anti-CD200R in Fallarino et al. versus CpG ODN in this study) and use of different methods to isolate DC populations and to assess their T cell stimulatory functions. Because IDO-dependent T cell suppressive functions of splenic CD19⁺ DCs were both potent and dominant (as well as exclusive) properties of these DCs, immunoregulatory outcomes could emerge when minor cohorts of CD19⁺ DCs are present among much larger cohorts of other DCs with T cell stimulatory functions.

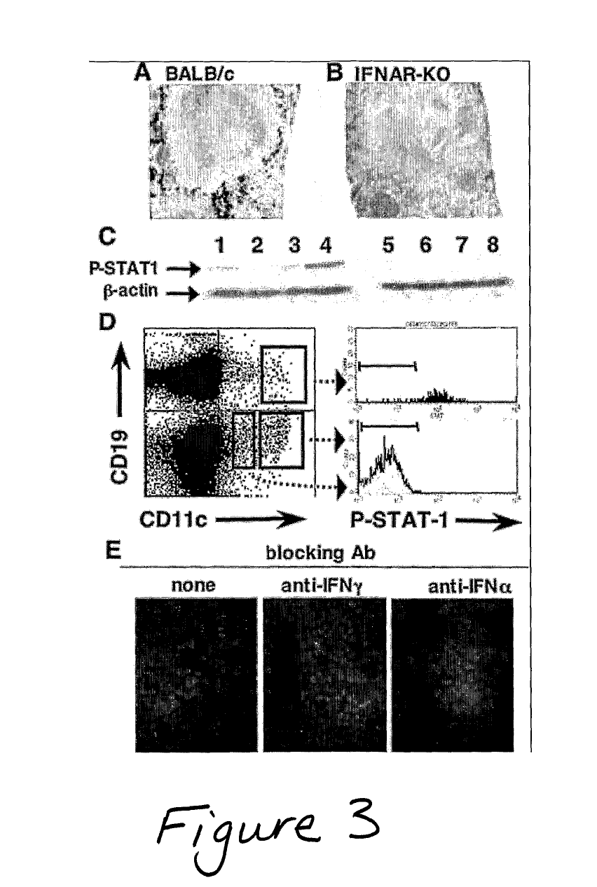

IFN-α signaling is essential for STAT-1 activation and IDO up-regulation in CD19⁺ DCs. To examine the mechanism of IDO up-regulation following CpG-ODN treatment, CpG-ODNs were injected into mice with defective IFN type I receptors (IFNAR-KO) and control BALB/c mice and assessed IDO expression in spleen as before. As in B6 mice, CpG-ODNs (no. 1826) induced IDO expression in spleen of BALB/c mice (FIG. 3A). In contrast, CpG-ODNs did not induce IDO up-regulation in spleens of IFNAR-KO mice (FIG. 3B). IFN-α signaling was also essential for STAT-1 activation because P-STAT-1 was not induced in splenocytes from IFNAR-KO (B6 background) mice exposed to CpG-ODNs, while STAT-1 activation was detected in splenocytes from treated IFN-γ R-KO (129/SvJ background) mice (FIG. 3C). This response was highly selective as P-STAT-1 was detected exclusively in CD19⁺ DCs following flow cytometric analyses (FIG. 3D).

Signaling requirements for STAT-1 activation were analyzed further by incubating CD11c⁺ DCs (AutoMACS enriched) from untreated BALB/c mice with CpG-ODNs (10 μg/ml) for five hours and staining cytospins to detect P-STAT-1. Activated PSTAT-1 was detected in ˜50% of DCs and had translocated to nuclei of ˜30% of DCs (FIG. 3E). This response was not observed when CD11c⁺ DCs were incubated with non-CpG ODNs at the same concentration. Addition of anti-IFN-γ mAb to cultures had no effect on STAT-1 activation induced by CpG-ODNs. However, STAT-1 activation was blocked completely when anti-IFN-α mAb was present. Collectively, these data revealed that IFN-α, but not IFN-γ, signaling was essential for STAT-1 activation and IDO up-regulation in CD19⁺ DCs following CpG-ODN treatment.

The outcomes reported above implied that IFN-α production occurred before selective STAT-1 activation in CD19⁺ DCs after CpG-ODN treatment. Based on previous studies (Asselin-Paturel et al., “Mouse type I IFN-producing cells are immature APCs with plasmacytoid morphology,” Nat. Immunol. 2001; 2:1144-1150; Corcoran et al., “The lymphoid past of mouse plasmacytoid cells and thymic dendritic cells,” J. Immunol. 2003; 170:4926-4932; Asselin-Paturel et al., “Type I interferon dependence of plasmacytoid dendritic cell activation and migration,” J. Exp. Med. 2005; 201:1157-1167; and Honda et al., “Spatiotemporal regulation of MyD88-IRF-7 signalling for robust type-I interferon induction,” Nature 2005; 434: 1035), pDCs are a likely source of IFN-α after CpG-ODN treatment. Purified CD11c^{high} and CD11c^{low} DCs secreted IFN-α rapidly after culture with CpG-ODNs (10 μg/ml, 5 hours). Because highly purified CD19⁺CD11c^{high} DCs up-regulated IDO after culture with CpG-ODNs (FIG. 1D), these DCs may also express sufficient IFN-α to induce STAT-1 activation and IDO up-regulation following TLR9 ligation (at least in vitro). Thus, IFN-α from pDCs may signal STAT-1 activation and subsequent IDO up-regulation in CD19 DCs via a paracrine mechanism, although the possibility that CD19⁺ DCs also produce IFN-α following CpG-ODN treatment cannot be excluded completely. Recent reports show that a cationic lipid N-[1-(2,3 dioleoyloxy)propyl]-N,N,N-trimethylammonium methylsulfate enhanced TLR9-mediated IFN-α production by DCs (Asselin-Paturel et al., J. Exp. Med. 2005; 201:1157-1167; and Honda et al., Nature 2005; 434:1035). Because N-[1-(2,3 dioleoyloxy) propyl]-N,N,N-trimethylammonium methylsulfate (DOTAP) was not required to induce IDO after CpG-ODN treatment, relatively low amounts of IFN-α may be sufficient to signal IDO up-regulation by CD19⁺ DCs. These issues notwithstanding, the key point is that rapid IFN-α production by DCs in response to TLR-9 ligation, whether autocrine or paracrine, is a critical mechanistic event that causes selective IDO induction in CD19⁺ DCs.

Microbial DNA containing unmethylated CpG motifs stimulates immune cell maturation and IFN-α production in humans and mice (Krieg et al., “CpG motifs in bacterial DNA trigger direct B cell activation,” Nature 1995; 374:546-549; Klinman, “Immunotherapeutic uses of CpG oligodeoxynucleotides,” Nat. Rev. Immunol. 2004; 4: 249-258; Krieg, “CpG motifs in bacterial DNA and their immune effects,” Annu. Rev. Immunol. 2002; 20:709-760). Classically, both events have been considered proinflammatory. However, it is not unusual in the immune system for proinflammatory stimuli to also elicit counterregulatory, anti-inflammatory responses that follow in short sequence. In mice, microbial infections, bacterial DNA containing CpG motifs, and synthetic CpG-ODNs induce a unique subset of splenic (B220⁺, CD11c^{low}, 120 G8⁺) pDCs to produce IFN-α (Asselin-Paturel et al., “Mouse strain differences in plasmacytoid dendritic cell frequency and function revealed by a novel monoclonal antibody,” J. Immunol. 2003; 171:6466-6477; Asselin-Paturel et al., Nat. Immunol. 2001; 2:1144-1150; and Corcoran et al., “The lymphoid past of mouse plasmacytoid cells and thymic dendritic cells,” J. Immunol. 2003; 170:4926-4932). In these (as in many other) previous studies, CD19 was used as an exclusion marker to remove B cells during purification of DC populations. Hence, to our knowledge, the effects of CpG-ODNs on CD19⁺(CD11c^{high}120 G8⁻) DCs identified in the current study, which accounted for <20% of all splenic DCs, have not been described previously. CD19⁺ DCs were the principal DC population to activate STAT-1, up-regulate IDO, and acquire potent and dominant IDO-dependent T cell-suppressive functions following i.v. administration of relatively high doses of CpG-ODNs to B6 mice. These responses by CD19⁺ DCs were dependent on IFN-α signaling, suggesting that CD19⁺ DCs may be specialized to respond to CpG-ODN-mediated TLR9 ligation by producing IFN-α themselves or by responding to IFN-α produced by pDCs in response to TLR9 ligation. The different anatomic locations of IDO⁺ CD19⁺ DCs in splenic red pulp and pDCs, which reside in T cell areas of secondary lymphoid tissues (Asselin-Paturel et al., J. Immunol. 2003; 171:6466-6477), testifies to the distinctive characteristics of these DC subsets, even though they share some morphologic (plasmacytoid) and phenotypic (B220⁺) characteristics. By ligating B7 molecules, CTLA4-Ig induced IFN-α-dependent STAT-1 activation and IDO up-regulation in a closely related (if not identical) population of splenic CD19⁺ DCs that coexpressed B220 and/or CD8α (Baban et al., Int. Immunol. 2005; 17: 909-919; and Mellor et al., Int. Immunol. 2004; 16:1391-1401). Thus, splenic CD19⁺ DCs are specialized to respond to IFN-α induced after TLR9 and B7 ligation by acquiring potent IDO-dependent T cell regulatory functions.

### Example 2

### CpG Oligonucleotides Enhance the Stimulatory Activity of IDO⁺ pDCs from Tumor Draining Lymph Nodes

Oligonucleotides containing unmethylated CpG motifs are potent stimulators of plasmacytoid DCs (Rothenfusser et al., “Plasmacytoid dendritic cells: the key to CpG,” Hum Immunol 2002; 63:1111), and have been shown to activate normally ineffectual tumor-associated DCs for enhanced T cell stimulation (Furumoto et al., “Induction of potent antitumor immunity by in situ targeting of intratumoral Dcs,” J Clin Invest 2004; 13:774; Lonsdorf et al., “Intratumor CpG-oligodeoxynucleotide injection induces protective antitumor T cell immunity,” J Immunol 2003; 171:3941; and Vicari et al., “Reversal of tumor-induced dendritic cell paralysis by CpG immunostimulatory oligonucleotide and anti-interleukin 10 receptor antibody,” J Exp Med 2002; 196:541). Therefore, this example addressed whether IDO⁺ pDCs were responsive to CpG (ODN-1826).

### Methods

B78H1-GMCSF tumors were grown in C57BL/6 mice. Tumors were implanted in female C57BL/6 mice (Jackson, Bar Harbor, Me.), 8-12 weeks of age. Mice were implanted with tumors in the anteriomedial thigh, using either 4×10⁴ B16F10 (ATCC, Bethesda, Md.) or 1×10⁶ B78H1 GM CSF (Huang et al., “Role of bone marrow-derived cells in presenting MHC class I-restricted tumor antigens,” Science 1994; 264:961).

For flow cytometry and cell sorting, single-cell suspensions of lymph nodes (LNs) were obtained by teasing and disaggregation through a 40 micron mesh; spleen cells were obtained by ground-glass homogenization and hypotonic lysis of erythrocytes. Dendritic cells (DC) were isolated from tumor-draining lymph nodes (TDLNs). Cells were stained by four color immunofluorescence, using CD11c versus B220 versus CD19 versus a panel of other markers. Fc binding was blocked using a commercial anti-CD16/CD32 cocktail (BD Pharmingen, San Diego, Calif.). Acquisition and sorting were performed using pulse-processing doublet discrimination on a Cytomation MoFlo cell sorter. Antibodies against the following antigens were from BD-Pharmingen: CD11c (clone HL3), B220 (clone RA3-6B2), CD19 (clone 1D3). Cells were fractionated into the CD19⁺ subset of plasmacytoid DCs (pDCs), isolated as CD11c⁺ B220⁺ CD19⁺ cells by flow cytometric sorting; the CD19^{NEG} pDCs (CD11c⁺ B220⁺ CD19^{NEG}); and all other CD11c⁺ B220^{NEG} DCs.

Each DC population was used as stimulators in mixed-leukocyte reactions (MLRs), with responder cells being BM3 T cells (TCR-transgenic, CD8⁺, recognizing H2K^{b} alloantigen on the DCs). T cell proliferation was assayed by four hour thymidine incorporation assay after three days. For measurement of T cell activation in MLR, TCR-transgenic BM3 responder T cells (CBA background, anti-H2K^{b} (Tarazona et al., “Effects of different antigenic microenvironments on the course of CD8⁺T cell responses in vivo,” Int Immunol 1996; 8:351)) were prepared from spleen by nylon-wool enrichment. Stimulator cells (sorted DCs) were mixed with 1×10⁵ BM3 responder cells at a ratio between 1:20 and 1:40 and cultured in 200 ul medium (10% fetal calf serum in RPMI-1640, with antibiotics and 50 uM 2-mercaptoethanol). All MLR assays were performed in quadruplicate. After three days, proliferation was measured by 4-hr tritiated-thymidine incorporation assay. All MLRs were performed in V-bottom culture wells (Nalge-Nunc, Rochester, N.Y.), as previously described (Munn et al., “Potential regulatory function of human dendritic cells expressing indoleamine 2,3-dioxygenase,” Science 2002; 297:1867), in order to ensure the close cell-cell contact required for optimal sensitivity to IDO-mediated suppression. Stimulator DCs were not irradiated, because preliminary validation studies showed that irradiation significantly altered the viability and functional attributes of IDO⁺ pDCs. MLRs were thus “two-way” reactions; however, the small number of sorted DCs used as stimulators contributed negligible proliferation compared to the large population of TCR-transgenic responder cells. Since the relevant readout was dominant suppression of all T cell proliferation, the two-way MLR design presented no problem in interpretation. Where indicated, replicate groups of wells received 200 uM 1-methyl-[D]-tryptophan (Sigma-Aldrich, St. Louis, Mo.). (Stock solution of 20 mM 1MT was dissolved in 0.1 N NaOH, then adjusted to pH 7.4.) Some experiments also received 5 uM CpG ODN-1826 (InvivoGen, San Diego Calif.). CpG was added at the beginning of the MLR.

### Results

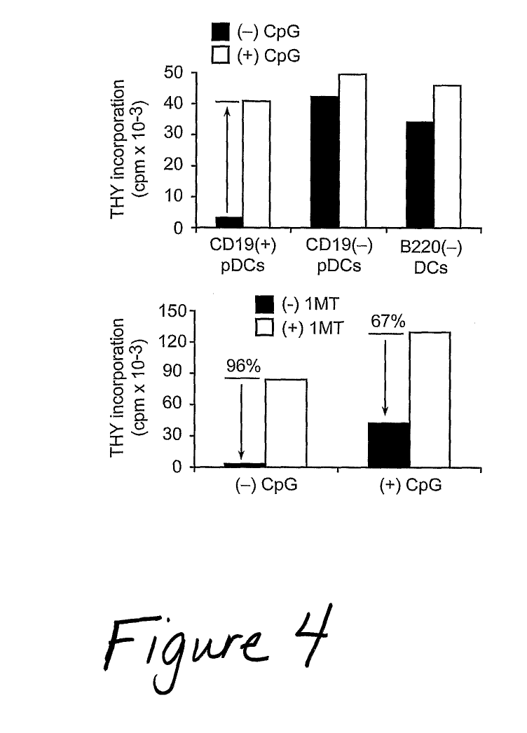

DCs were isolated by FACS sorting cells from TDLNs of B78H1-GMCSF tumors, as described (Munn et al, “Expression of indoleamine 2,3-dioxygenase by plasmacytoid dendritic cells in tumor-draining lymph nodes,” J Clin Invest 2004; 114:280), based on the following markers: CD11c⁺B220⁺CD19^{NEG} (conventional IDO^{NEG} pDCs), CD11c⁺B220⁺CD19⁺ (IDO⁺ pDCs), and CD11c⁺B220^{NEG} (non-plasmacytoid DCs). These different DC populations were used to stimulate BM3 T cells (TCR-transgenic, recognizing H2K^{b} as an alloantigen on the target DCs). FIG. 4A shows that CpG had a marked effect on the ability of CD19⁺ pDCs, increasing their stimulatory activity from a very low level to a high level comparable with the other non-suppressive DCs. In contrast, CpG had much less effect on the CD 19^{NEG} pDCs or on the non-plasmacytoid DCs, both of which were already stimulatory. Further experiments, using CpG combined with 1 MT (FIG. 4B), revealed that the degree to which CpG enhanced the stimulatory activity of the CD19⁺ pDCs was markedly increased when IDO was also blocked by 1 MT (open bars). Thus, CpG allowed at least some T cell proliferation to occur in response to the normally potently suppressive IDO⁺ pDCs even when IDO was active, and a but a much greater T cell response to these same IDO⁺ pDCs when IDO was blocked.

As shown in FIG. 4A, CD19⁺ pDCs (which comprised the cells mediating IDO-dependent suppression) stimulated little proliferation of the responder T cells, compared with the other, non-suppressive DCs (black bars). An increase in proliferation of responder cells was seen when the CD19⁺ pDCs were treated with CpG (1826), as shown by the arrow. CpG allowed T cell proliferation in the group stimulated by CD19⁺ pDC comparable to that seen with the other, non-suppressive DCs in the same experiment. The largest magnitude of response to CpG was seen with the CD19⁺ pDCs. However, as shown in the FIG. 4B, a much greater response could be obtained if CpG was combined with 1 MT. The upper and lower panels are taken from the same experiment with the same responder cells, so the absolute counts can be meaningfully compared. The lower panel is graphed on a larger scale because the counts were much greater. The lower panel shows MLRs using only the CD19⁺ pDCs as stimulators. MLRs were performed with and without CpG added to the MLRs as shown, and with or without 200 uM 1 MT (D-isomer). The right-hand set of bars shows that the enhanced T cell proliferation obtained by adding CpG to the MLRs could be increased another 3-fold by inhibiting IDO with 1MT. Thus, the combination of CpG plus 1 MT produced an effect on T cells that was greater than that produced by either agent alone.

### Example 3

### In Vivo Administration of 1 MT and CpG in EG7 Tumor Model

### Methods

C57BL/6 (B6) mice were from Jackson Laboratory (Bar Harbor, Me.). Mice were implanted with tumors in the flank, using 1×10⁶ E.G7 mouse lymphoma tumor cells (American Type Culture Collection, Bethesda, Md.). Administration of 1 MT by implantable subcutaneous pellets was performed as previously described (Munn et al., “Prevention of allogeneic fetal rejection by tryptophan catabolism,” Science 1998; 281:1191), using slow-release polymer pellets impregnated with 1-methyl-D-tryptophan (5 mg/day for 14 days) or vehicle control pellets inserted surgically under dorsal skin. Pellets were implanted on day one after tumor implantation. Beginning day two after tumor implantation and weekly thereafter (days 2, 9, 16 and 23) mice received CpG 1826 (Ballas et al., “Divergent therapeutic and immunologic effects of oligodeoxynucleotides with distinct CpG motifs,” J Immunol 2001; 167:4878) at a dose of 100 ug intraperitoneally, or saline control. Each group comprised five mice. Tumor area was measured as the product of orthogonal diameters.

### Results

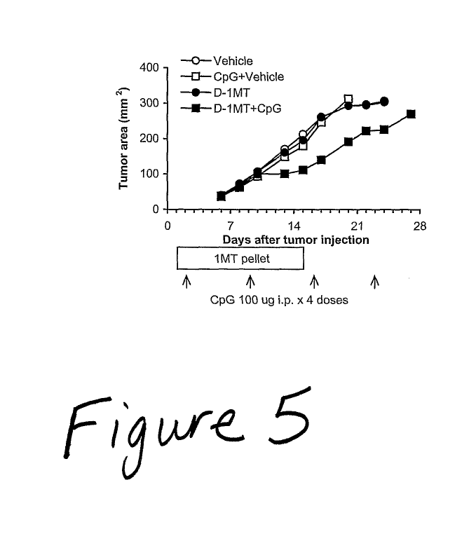

As shown in FIG. 5, the murine E.G7 lymphoma grew aggressively in syngeneic B6 hosts, usually reaching the ethical surrogate endpoint size of 300 mm² within approximately three weeks. Treatment for two weeks with continuous infusion of D-1 MT had no effect on tumor growth. Likewise, treatment with CpG 1826 (Ballas et al., J Immunol 2001; 167:4878) 100 ug i.p. weekly for four doses had no detectable effect on tumor growth using this regimen. However, as shown in FIG. 5, the combination of 1 MT and CpG showed a delay in tumor growth, and an increased time to reach the surrogate endpoint

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

For any method disclosed herein that includes discrete steps, the steps may be conducted in any feasible order. And, as appropriate, any combination of two or more steps may be conducted simultaneously.

1. A method of enhancing an immune response in a subject, the method comprising administering to the subject a TLR9 agonist and an inhibitor of IDO.

2. The method of claim 1, the method further comprising administering to the subject an antigen and wherein the immune response to the antigen in the subject is enhanced.

3. The method of claim 1, wherein CD8 T cell activation in the subject is stimulated.

4. A method of treating cancer in a subject, the method comprising administering to the subject a TLR9 agonist, an inhibitor of IDO, and an additional antineoplastic therapeutic agent.

5. The method of claim 6, wherein a T cell mediated immune response in the subject is suppressed.

6. A method of inducing indoleamine 2,3-dioxygenase (IDO) expression in dendritic cells in a subject, the method comprising administering to the subject a TLR9 agonist in an amount effective to induce IDO expression in a subset of dendritic cells.

7. (canceled)

8. A method of suppressing T cell activation to an antigen in a subject, the method comprising administering to the subject a TLR9 agonist and the antigen, wherein the TLR9 agonist is administered in an amount effective to induce IDO expression in a subset of dendritic cells.

9. The method of claim 8 wherein the TLR9 agonist and the antigen are administered simultaneously.

10. The method of claim 8 wherein the TLR9 agonist is administered prior to the administration of the antigen.

11. The method of claim 8, wherein the antigen is an alloantigen.

12. A method of inducing tolerance to an antigen in a subject, the method comprising administering to the subject a TLR9 agonist and the antigen, wherein the TLR9 agonist is administered in an amount effective to induce IDO expression in a subset of dendritic cells.

13. A method of preventing allograft rejection in a subject, the method comprising administering to the subject a TLR9 agonist and one or more alloantigens present in the allograft.

14. The method of claim 13 wherein the TLR9 agonist is administered to the recipient after the transplantation of the allograft into the recipient.

15. A method of preventing graft versus host disease in a recipient, the method comprising:
administering to the donor a TLR9 agonist and one or more alloantigens present in the recipient, wherein the TLR9 agonist and the one or more alloantigens present in the recipient are administered to the donor prior to obtaining donor cells from the donor;
obtaining donor cells from the donor; and
administering the donor cells to the recipient.

16. A method of preconditioning a recipient of an allograft to suppress allograft rejection in the recipient, the method comprising:
administering to the recipient a TLR9 agonist and one or more alloantigens present in the allograft, wherein the TLR9 agonist and the one or more alloantigens present in the allograft are administered to the recipient prior to allografting; and
transplanting the allograft into the recipient.

17. The composition of claim 18, the composition further comprising an antigen.

18. A composition comprising a TLR9 agonist and an inhibitor of IDO.

19. An isolated cell population preconditioned to minimize graft versus host disease when transplanted into a donor recipient, the cell population obtained by a method comprising:
administering to the donor a TLR9 agonist and one or more alloantigens present in the recipient, wherein the TLR9 agonist and the one or more alloantigens present in the recipient are administered to the donor prior to obtaining donor cells from the donor; and
obtaining donor cells from the donor.

20. The method of claim 1, wherein the TLR9 agonist is a CpG-oligonucleotide

21. The method of claim 1, wherein the TLR9 agonist is administered systemically.


SEQUENCE LISTING (2 sequences)

SEQ ID NO: 1 - DNA, 20 bp, Artificial Sequence
  Commercially Available CpG oligodeoxynucleotide
tccatgacgttcctgacgtt

SEQ ID NO: 2 - DNA, 20 bp, Artificial Sequence
  Commerically available CpG Oligodeoxynucleotide
tccatgagcttcctgagctt